(19) United States
(12) Patent Application Publication
(10) Pub. No.: US 2010/0284957 A1
(43) Pub. Date: Nov. 11, 2010

(54) Powder Treated with (Meth)Acrylic-Grafted Silicone Polymer and Product Using the Same

(76) Inventors: Takeshi Yamada, Kanagawa (JP); Saloshi Haramizu, Tokyo (JP)

Correspondence Address: 3M INNOVATIVE PROPERTIES COMPANY, PO BOX 33427, ST. PAUL, MN, 55133-3427, US

(21) Appl. No.: 12/810,233

(22) PCT Filed: Dec. 22, 2008
(86) PCT No.: PCT/US2008/087931

§ 371 Date: Jun. 23, 2010

(30) Foreign Application Priority Data
Dec. 28, 2007 (JP) 2007-339836

(51) Int. Cl. A61K 8/899; A61Q 19/00; A61Q 1/00

(52) U.S. Cl. 424/78.03

(57) ABSTRACT

A powder treated with a (meth)acrylic-grafted silicone polymer, the treated powder being obtained from a mixture containing a volatile silicone oil, a (meth)acrylic-grafted silicone polymer in which a monomer component comprising a (meth)acrylic series monomer has been grafted onto the main chain of a polysiloxane, and a powder.

### BACKGROUND

Powders for cosmetic products are mixed into various types of cosmetics for such purposes as coloring the skin, hair, and nails; concealing wrinkles and freckles; protecting the skin from ultraviolet rays; and so on. Such powders for cosmetic products include, for example, inorganic pigment powders and polymer powders.

Inorganic pigments generally exhibit poor dispersibility in an oil phase due to the hydrophilic surfaces of the inorganic pigments. The poor dispersibility can lead to an increase in viscosity, color separation, aggregation of particles, precipitation, and the like. Therefore, attempts have been made to treat the surface of the powder (for example, adsorption of a metallic soap onto the inorganic pigment) to improve the dispersibility thereof in the oil phase. When the surface treatment of the powder is performed using silicone, the surface-treated powder obtained thereby not only has increased dispersibility in the oil phase, but also has improved water repellency and adhesion to the skin.

The use in cosmetic products of pigment or extender pigment that has been surface-treated with an organosilicon compound (particularly, methyl hydrogen polysiloxane) is well-known as referenced in Japanese Patent Application Laid-open No. H7-196946.

Also, an organosilicon compound-treated pigment or extender pigment has been disclosed wherein a straight chain, reactive alkyl polysiloxane having a degree of polymerization of 25 to 100, having a ratio (Mw/Mn) of the weight-average molecular weight (Mw) to the number-average molecular weight (Mn) of 1.0 to 1.3, and having an amino hydrogen group, halogen atom, hydroxyl group, or alkoxy group on one end of the molecular chain thereof is oriented and adhered to the surface of a pigment or extender pigment by a heat treatment (Japanese Patent Application Laid-open No. 7-196946).

In addition, Japanese Patent Application Laid-open No. 5-339125 discloses a powder for a cosmetic product and a cosmetic material containing that powder. The powder for a cosmetic product is obtained by subjecting a powder to a coating treatment in a fluid bed coating process using an acrylic-silicone based graft copolymer formed by radical copolymerization of (A) a dimethyl polysiloxane compound having a radical reactive group on one end of the molecular chain thereof and (B) an alkyl(meth)acrylate, or by dissolving that copolymer in a volatile solvent, mixing and dispersing the powder therewith to form a slurry, and heating the slurry under reduced pressure to remove the solvent.

Despite the aforementioned progress in powder surface treatment, a powder for a cosmetic product is needed that provides even better coverage and texture on the skin when contained in a cosmetic product, without loss of water repellency and dispersibility in the oil phase.

### SUMMARY

In the present specification, a powder treated with a (meth)acrylic-grafted silicone polymer is obtained from a mixture containing a volatile silicone oil, a (meth)acrylic-grafted silicone polymer in which a monomer component comprising a (meth)acrylic series monomer has been grafted onto the main chain of a polysiloxane, and a powder.

Moreover, a cosmetic product containing the aforementioned treated powder is provided in the present specification.

In accordance with the present specification it has become possible to obtain a powder for a cosmetic product that has excellent water repellency and dispersibility in the oil phase, and that can provide better coverage and texture on the skin when contained in a cosmetic product.

Moreover, even if the amount of the (meth)acrylic-grafted silicone polymer used for the treatment of the powder is very small, when a cosmetic product contains the obtained treated powder, coverage and texture on the skin can be greatly improved in comparison with an untreated powder.

### DETAILED DESCRIPTION

The present invention relates to a powder treated with a (meth)acrylic-grafted silicone polymer and a product using the same, particularly a personal care product containing a cosmetic.

In the present specification, the term “(meth)acrylic-grafted silicone polymer” refers to an “acrylic-grafted silicone polymer” or a “methacrylic-grafted silicone polymer” corresponding thereto; and the terms “(meth)acrylic series monomer,” “(meth)acrylic acid,” and “(meth)acrylic acid ester” respectively refer to an “acrylic monomer,” “acrylic acid,” or “acrylic acid ester” or to a “methacrylic monomer,” “methacrylic acid,” or “methacrylic acid ester” corresponding thereto.

In the present specification the powder treated with a (meth)acrylic-grafted silicone polymer is obtained from a mixture containing a volatile silicone oil, a (meth)acrylic-grafted silicone polymer in which a monomer component comprising a (meth)acrylic series monomer has been grafted onto the main chain of a polysiloxane, and a powder.

A volatile silicone oil is used in the mixture from the standpoint of water repellency, coverage on the skin, and texture of the obtained treated powder. The present invention does not particularly limit the volatile silicone oil, and any volatile silicone oil can be used therein provided it can dissolve the (meth)acrylic-grafted silicone polymer and can be removed in the process of producing the treated powder. Examples of volatile silicone oils include hexamethyl disiloxane, octamethyl trisiloxane, decamethyl tetrasiloxane, dodecamethyl pentasiloxane and other straight chain silicone oils; methyl trimethicone and other branched silicone oils; caprylyl-modified trisiloxane and other modified silicone oils; and hexamethyl cyclotrisiloxane, octamethyl cyclotetrasiloxane, decamethyl cyclopentasiloxane, dodecamethyl cyclohexasiloxane and other cyclic silicone oils; and the like. These can be used alone or in combinations of two or more types thereof. Herein, the term “volatile” refers to the property of easily evaporating into the air at normal temperatures and more particularly, having a boiling point from 50° C. to 240° C. In one embodiment of the present invention, the use of dimethyl polysiloxane and/or decamethyl cycloheptasiloxane is preferred from the standpoint of workability (for example, rate of evaporation of the volatile silicone oil, boiling point, freezing point, flash point, viscosity, stability, etc.) in the process step of obtaining the powder treated with the (meth)acrylic-grafted silicone polymer.

Next, (meth)acrylic-grafted silicone polymer will be described. The (meth)acrylic-grafted silicone polymer has a structure in which a monomer component comprising a (meth)acrylic series monomer has been grafted onto the main chain of a polysiloxane.

The (meth)acrylic-grafted silicone polymer may have the structure shown in General Formula (2) shown below.

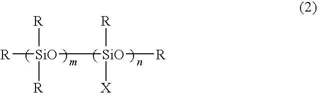

In the above formula, each R independently represents a hydrogen atom, hydroxyl group, alkyl group, allyl group, halogenated alkyl group, or arylene group; and X is the structural member in which the monomer component comprising a (meth)acrylic series monomer is grafted. Furthermore, m is an integer from 10 to 540, and n is an integer of 1 or more. In Formula (2) above (and also in Formulas (1), (3), and (4) herein), the moieties —(RRSiO)ₘ— and —(RXSiO)ₙ— forming the main chain of the polysiloxane may be arranged in block format or randomly distributed.

In some embodiments of the present invention, each R in Formula (2) above independently represents a hydrogen atom, hydroxyl group, allyl group, alkyl group of 1 to 3 carbons, or a halogenated alkyl group of 1 to 3 carbons. In other embodiments of the present invention, each R independently represents a hydroxyl group, allyl group, or alkyl group of 1 to 3 carbons. Preferably each R independently represents an alkyl group of 1 to 3 carbons, and more preferably a methyl group. Additionally, it is especially preferred if all R moieties are methyl groups.

In some embodiments of the present invention, the weight-average molecular weight of the polysiloxane main chain moiety can be set at 750 or more, and from the standpoint of the viscoelasticity of the obtained (meth)acrylic-grafted silicone polymer, a weight-average molecular weight of 3,500 or more is even more preferred. Moreover, from the standpoint of solubility in the volatile silicone oil, the weight-average molecular weight of the polysiloxane main chain moiety is preferably 40,000 or less, and more preferably 25,000 or less.

The amount of component X, i.e., the monomer component comprising the (meth)acrylic series monomer, that is grafted onto the polysiloxane main chain can be set within the range of 30 to 300 parts by weight per 100 parts by weight of the polysiloxane main chain. From the standpoint of the viscoelasticity of the obtained (meth)acrylic-grafted silicone polymer, the content of monomer component comprising a (meth)acrylic series monomer is preferably 40 parts by weight or more per 100 parts by weight of polysiloxane main chain. Moreover, from the standpoint of the solubility in the volatile silicone oil, a content of 200 parts by weight or less per 100 parts by weight of polysiloxane main chain is preferred.

Examples of a (meth)acrylic series monomer that can be grafted onto the polysiloxane main chain include an acidic (meth)acrylic series monomer, basic (meth)acrylic series monomer, hydroxyl group-containing (meth)acrylic series monomer, (meth)acrylic acid ester type (meth)acrylic series monomer, and the like, but the present invention is not limited thereto. In addition, these (meth)acrylic series monomers may be grafted as a homopolymer or may be grafted as a copolymer comprising a plurality of (meth)acrylic series monomers.

Examples of an acidic type (meth)acrylic series monomer include (meth)acrylic acid, crotonic acid, ethacrylic acid, propyl(meth)acrylic acid, isopropyl(meth)acrylic acid, itaconic acid, fumaric acid, (meth)acryloyloxy ethyl phthalate, (meth)acryloyloxy succinate, (meth)acrylic acid-2-ethyl sulfonate, butyl(meth)acrylamide sulfonic acid, (meth)acrylic acid-2-ethyl phosphonate, and the like.

Examples of a basic type (meth)acrylic series monomer include (meth)acrylic acid amide, amino ethyl(meth)acrylate, amino propyl(meth)acrylate, methyl amino ethyl (meth)acrylate, methyl amino propyl(meth)acrylate, ethyl amino ethyl(meth)acrylate, ethyl amino propyl(meth)acrylate, dimethyl amino ethyl(meth)acrylate, diethyl amino ethyl(meth)acrylate, dimethyl amino propyl(meth)acrylate, diethyl amino propyl (meth)acrylate, (meth)acrylic acid dimethyl amino ethyl methyl chloride salt and (meth)acrylic acid dimethyl amino ethyl benzyl chloride salt, and the like.

Examples of a hydroxyl group-containing (meth)acrylic series monomer include 2-hydroxyethyl(meth)acrylic acid, hydroxypropyl(meth)acrylic acid, hydroxybutyl (meth)acrylic acid, and the like.

Examples of a (meth)acrylic acid ester type (meth)acrylic series monomer include methyl(meth)acrylate, ethyl(meth)acrylate, isopropyl(meth)acrylate, n-propyl (meth)acrylate, n-butyl(meth)acrylate, t-butyl(meth)acrylate, octyl(meth)acrylate, 2-ethyl hexyl(meth)acrylate, decyl(meth)acrylate, cetyl(meth)acrylate, stearyl (meth)acrylate, behenyl(meth)acrylate and other alkyl esters of (meth)acrylic acid; cyclohexyl(meth)acrylate, isobornyl(meth)acrylate and other non-aromatic cyclic esters of (meth)acrylic acid (alicyclic alkyl esters and cycloalkyl esters); 2-methoxy ethyl (meth)acrylate, 2-ethoxy ethyl(meth)acrylate, and other alkoxy esters of (meth)acrylic acid; benzyl(meth)acrylate and other aryl esters of (meth)acrylic acid; and the like.

Monomer components other than the (meth)acrylic series monomer that can be grafted to the polysiloxane main chain are not particularly limited by the present invention provided they are copolymerizable with the (meth)acrylic series monomer. Examples include ethylene, propylene, styrene, vinyl acetate, vinyl ether, vinyl chloride (meth)acrylonitrile, N-vinyl pyrrolidone, butadiene, isoprene, and the like. The amount of this kind of monomer component other than the (meth)acrylic series monomer will differ depending on the monomer components to be used, but a desired amount can be included therein provided it is within a range such that the properties of the obtained powder treated with the (meth)acrylic-grafted silicone polymer are not lost. Generally, the amount of a monomer component other than the (meth)acrylic series monomer will preferably be no more than half the amount of all monomer components grafted to the polysiloxane main chain.

In some embodiments of the present invention, the monomer component comprises at least one type of (meth)acrylic series monomer selected from the group consisting of (meth)acrylic acid and (meth)acrylic acid esters. In other embodiments of the present invention, the monomer component is a (meth)acrylic acid and/or (meth)acrylic acid ester. Furthermore, from the standpoint of solubility in the volatile silicone oil, the monomer component preferably comprises a (meth)acrylic acid ester. The (meth)acrylic-grafted silicone polymer can be obtained by grafting the monomer component comprising the (meth)acrylic series monomer to a silicone polymer containing the radical-reactive group shown in General Formula (3) below wherein R, m, and n, are defined as above, and Z is a radical-reactive group.

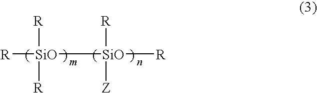

In some embodiments of the present invention, the radical-reactive group Z is a mercaptoalkyl group (i.e., a mercaptoalkyl-modified silicone polymer). The mercapto-modified silicone polymer can be prepared by any desired publicly known method such as: (1) simultaneous hydrolysis of a mixture of an organo alkoxy silane having 1 or a plurality of mercapto-substituted hydrocarbon groups and an organo alkoxy silane having no mercapto groups, (2) reaction of an organo alkoxy silane having 1 or a plurality of mercapto-substituted hydrocarbon groups with a cyclic organo polysiloxane or a silanol-terminated diorgano polysiloxane having no mercapto groups, (3) equilibrium reaction of a cyclic or straight-chain organo polysiloxane having 1 or a plurality of mercapto-substituted hydrocarbon groups with a cyclic or straight-chain organo polysiloxane having no mercapto groups, (4) reaction of 1 or a plurality of nucleophilic groups with an electrophilic reagent to obtain a mercapto-modified silicone polymer, and (5) reaction of 1 or a plurality of electrophilic groups with a nucleophilic reagent to obtain a mercapto-modified silicone polymer.

The mercapto-modified silicone polymer can be purchased as a commercial product. Examples include KF-2001™ and KF-2004™ manufactured by Shin-Etsu Chemical Co., Ltd.; SMS-022™, SMS-042™ and SMS-992™ manufactured by Gelest, Inc.; and PS848™, PS849™, PS849.5™, PS850™, PS850.5™ and PS927™ manufactured by United Chemical Technologies. These commercial products each differ with respect to weight-average molecular weight, molecular weight distribution, and mercapto group ratio, and they can be selected as desired.

In some embodiments of the present invention, the (meth)acrylic-grafted silicone polymer comprises a polymerization product between a mercapto-modified silicone polymer represented by General Formula (1) below and a monomer component comprising at least one type of (meth)acrylic series monomer selected from the group consisting of (meth)acrylic acid and (meth)acrylic acid esters:

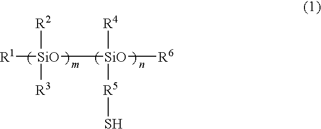

wherein R¹, R², R³, R⁴, and R⁶ each independently represents a hydrogen atom, hydroxyl group, allyl group, alkyl group of 1 to 3 carbons, or a halogenated alkyl group of 1 to 3 carbons; R⁵ represents an arylene group or alkylene group of 1 to 3 carbon atoms; m is an integer from 10 to 540; and n is an integer of 1 or more.

In other embodiments of the present invention the mercapto-modified silicone polymer is represented by General Formula (4) below wherein m is an integer from 10 to 540, and n is an integer of 1 or more.

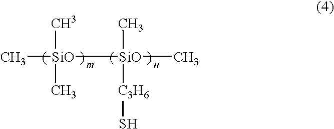

The (meth)acrylic-grafted silicone polymer can be obtained specifically by polymerization of the above silicone polymer containing a radical-reactive group and the above monomer component in which the monomer is grafted onto the silicone polymer containing the radical-reactive group via the radical-reactive group thereof in the presence of a radical polymerization initiator. The present invention does not particularly limit the radical polymerization initiator. Azo compounds, peroxide compounds, hydroxyperoxide compounds, peroxy acid compounds and per ester compounds can be used as a thermal polymerization initiators. Examples of an azo compound include 2,2′-azo bis-isobutyronitrile, 2,2′-azo bis-(2-methyl butyronitrile), dimethyl 2,2′-azo bis-isobutyrate, dimethyl 2,2′-azo bis-(2-methyl propionate), azo bis-diphenyl methane, and 4,4′-azo bis-(4-cyano pentanoic acid). Examples of the peroxide compound include benzoyl peroxide, cumyl peroxide, tert-butyl peroxide, cyclohexanone peroxide, glutaric acid peroxide, lauroyl peroxide, methyl ethyl ketone peroxide, and hydrogen peroxide. Examples of the hydroxy peroxide compound include tert-butyl hydroperoxide and cumene hydroperoxide. Examples of the peroxy acid compound include peracetic acid, perbenzoic acid, and potassium persulfate. Examples of the per ester compound include diisopropyl percarbonate and the like. In addition, a benzoin ether such as diethoxy acetophenone, oximinoketone, acyl phosphone oxide, diaryl ketone, benzophenone, 2-isopropyl thioxantone, benzil, quinone derivatives, and 3-ketocumarin can be used as a photoinitiator. These compounds may be used alone or as a mixture of 2 or more thereof.

Solution polymerization performed in a solvent is preferred as the polymerization method thereof. A desired organic solvent that is inert with respect to the monomer and the reaction product in the radical polymerization reaction, and that does not have a detrimental effect on the reaction is preferred as the solvent used in solution polymerization. Moreover, a solvent that is a liquid at temperatures between −10° C. and 50° C. is preferred. More specifically, an ester solvent, ketone solvent, and alcohol solvent is preferred. Specific examples of these solvents include ester solvents such as ethyl acetate and butyl acetate; ketone solvents such as acetone, methyl ethyl ketone, methyl isobutyl ketone, and methyl propyl ketone; and alcohol solvents such as methanol, ethanol, isopropanol and butanol.

When the polymerization reaction is performed, first the silicone polymer containing a radical-reactive group, the monomer component comprising the (meth)acrylic monomer, the radical polymerization initiator, and the solvent are placed in a desired container, and the radical polymerization initiator is dissociated by light or heat to carry out radical polymerization. The (meth)acrylic-grafted silicone polymer produced thereby can be recovered by evaporation of the solvent and by precipitation in a suitable solvent (for example, methanol, hexane, or water). Moreover, the obtained (meth)acrylic-grafted silicone polymer may be a mixture of polymers with different degrees of polymerization, polymer ends, and the like.

Next, the powder will be described. Any powder known to prior art may be used as the powder without limitation. In some embodiments of the present invention, a powder for cosmetic product usage is used. Specific examples include an inorganic pigment, organic pigment, compound powder of inorganic pigment and/or inorganic pigment, polymer powder, pearl pigment, luster pigment, and the like. It is also possible to combine and use a plurality of these powders.

Examples of an inorganic pigment include titanium dioxide, zinc oxide, cerium oxide, red iron oxide, yellow iron oxide, black iron oxide, hydrous iron oxide, ocher, mango violet, cobalt violet, aluminum oxide, magnesium oxide, chromium oxide, chromium hydroxide, cobalt titanium oxide, ultramarine blue, Prussian blue, zirconium dioxide, talc, kaolin, sericite, muscovite, phlogopite, lepidolite, synthetic mica, lithium mica, vermiculite, magnesium carbonate, calcium carbonate, diatomaceous earth, magnesium trisilicate, calcium silicate, aluminum silicate, silica, hydroxyapatite, zeolite, boron nitride, barium sulfate, and the like.

Examples of an organic pigment include Red #201 (lithol rubine B), Red #202 (lithol rubine BCA), Red #203 (lake red C), Red #204 (lake red CBA), Red #207 (lithol red), Red #213 (rhodamine B), Red #226 (Helindone pink CN), Red #405 (permanent red), Blue #1 (brilliant blue FCF), Blue #404 (phthalocyanine blue), Yellow #4 (tartrazine), Yellow #5 (sunset yellow FCF), saffron, β-carotene, chlorophyl, cochineal extract, and the like.

Examples of a compound powder include titanium dioxide-iron oxide, titanium dioxide-silica, sericite-titanium dioxide, silica-cerium oxide, silica-zinc oxide, mica-aluminum hydroxide, mica-titanium dioxide-silica, mica-iron oxide-silica, titanium oxide-iron oxide-silica, barium sulfate-titanium dioxide, barium sulfate-zinc oxide, mica-barium sulfate-titanium dioxide, mica-barium sulfate-zinc oxide, and the like.

Examples of a polymer powder include nylon powder, silicone powder, polyethylene powder, benzoguanamine powder, polytetrafluoroethylene powder, polystyrene powder, cellulose powder, urethane powder, polyethylene-polymethacrylic acid powder, epoxy powder, polymethacrylic acid powder and the like.

Examples of a pearl powder and luster powder include fish scale foil, aluminum powder, gold-coated glass flakes, metal oxide coated glass flakes, polyethylene terephthalate-aluminum-urethane laminate powder, polyethylene terephthalate-polymethyl methacrylate laminate powder, and the like.

Even if a powder is treated with only a small amount of the (meth)acrylic-grafted silicone polymer, the powder obtained thereby has a considerable effect in improving coverage and texture. In one embodiment of the present invention, the amount of (meth)acrylic-grafted silicone polymer in the mixture can be set at 0.1 parts by weight or more per 100 parts by weight of powder. To provide more stable coverage and texture, the amount of (meth)acrylic-grafted silicone polymer can be set at 0.5 parts by weight or more, and more preferably 1 part by weight or more, per 100 parts by weight of powder. The upper limit of the (meth)acrylic-grafted silicone polymer is not particularly limited in the present invention, but generally, if more than a set amount is used in the treated powder obtained thereby, the improving effect on coverage and texture changes little. In some embodiments of the present invention, the amount of (meth)acrylic-grafted silicone polymer can be set at 10 parts by weight or less per 100 parts by weight of powder.

In addition to the aforementioned three components other ingredients may be contained in the mixture. Specific examples include: a volatile solvent other than the volatile silicone oil, plasticizer, surfactant, film-forming agent, powder treatment agent, oil, and the like.

The present invention does not particularly limit a volatile solvent other than the volatile silicone oil provided it is capable of dissolving the (meth)acrylic-grafted silicone polymer. For example, an aromatic hydrocarbon such as benzene, toluene, xylene, and the like; ketone such as methyl ethyl ketone, methyl isobutyl ketone, and the like; ester such as ethyl acetate, isobutyl acetate, and the like; and alcohol such as isopropanol, butanol, and the like can be used. From the standpoint of coverage and texture of the treated powder obtained thereby, the amount of volatile solvent other than the volatile silicone oil will preferably be minimal, and more preferably an amount less than the amount of the volatile silicone oil used therewith. Particularly preferred is a mode wherein no volatile solvent other than the volatile silicone oil is contained therein.

Next, the method of treating the powder with the (meth)acrylic-grafted silicone polymer will be described. As the processing method first a mixture of the volatile silicone oil, (meth)acrylic-grafted silicone polymer, and the powder is prepared. During this procedure, a mixture can be prepared by dissolving the (meth)acrylic-grafted silicone polymer in the volatile silicone oil, adding the powder to this solution, and stirring. In preparing the mixture, the aforementioned optional ingredients (a volatile solvent other than the volatile silicone oil, plasticizer, surfactant, film forming agent, powder treatment agent, oil and the like) can be suitably added.

Next, the removal of the volatile components (including the volatile silicone oil) from the obtained mixture and the drying process thereof are performed. The removal of volatile components and drying can be performed at room temperature, and these processes can also be performed at a higher temperature (for example, approximately 50° C. to 100° C.) and under reduced pressure. The removal and the drying time can be set appropriately. In general, it will last for 30 to 360 minutes.

The powder treated with the (meth)acrylic-grafted silicone polymer can be mixed into a cosmetic product. Here, when the powder treated with the (meth)acrylic-grafted silicone polymer is compared with a powder treated in the same manner with the same amount of silicone-grafted (meth)acrylic polymer (polymer having a silicone-grafted member on a (meth)acrylic polymer main chain), the powder treated with the (meth)acrylic-grafted silicone polymer is superior to the powder treated with the silicone-grafted (meth)acrylic polymer from the standpoint of coverage and texture on the skin. This difference is especially remarkable in an actual cosmetic product mixture, and when the powder treated with the (meth)acrylic-grafted silicone polymer is used, a cosmetic product can be obtained that has excellent coverage, adhesion to the skin, and blending properties, and it does not lose its fresh appearance over time. Thus, the powder treated with the (meth)acrylic-grafted silicone polymer is very suitable for use in a cosmetic product. Moreover, because the powder treated with the (meth)acrylic-grafted silicone polymer has a considerable improving effect on coverage and texture even when the powder is treated with only a small amount thereof, from the standpoint of an actual cosmetic product formulation, sufficient cosmetic properties can be obtained by using only a small amount of the (meth)acrylic-grafted silicone polymer. This becomes a major advantage from the standpoint of production on a commercial scale and product cost.

Examples of types of cosmetic products using the powder treated with the (meth)acrylic-grafted silicone polymer include: skin care cosmetics such as facial cleansing creams, facial packs, and the like; sun care cosmetics such as sunscreen and the like; and makeup cosmetics such as makeup base, foundation, concealer, face powder, lipstick, lip gloss, lip cream, rouge, mascara, eye shadow, eyebrow makeup, eyeliner, and the like.

Examples of the form of the cosmetic include: a solution, gel, bilayer, water-in-oil emulsion, oil-in-water emulsion, oil, spray, mousse, powder, solid, and the like.

The content of the powder added to the cosmetic product will differ depending on the intended purpose thereof. Generally, per 100 parts by weight of cosmetic product, the amount of powder included can be set as follows: 70 to 95 parts by weight for powder foundation; 5 to 60 parts by weight for liquid foundation; 20 to 80 parts by weight for oil-based foundation; 80 to 100 parts by weight for face powder; 5 to 30 parts by weight for lipstick; 70 to 95 parts by weight for solid rouge; and 5 to 30 parts by weight for a sunscreen cosmetic product.

Moreover, when the powder treated with the (meth)acrylic-grafted silicone polymer is mixed into a cosmetic product, it can be freely combined with the ingredients (cosmetic raw materials) usually used for cosmetic products according to the type and the formulation of the cosmetic product. The following are noted as specific ingredients.

Hydrocarbons: Examples include liquid paraffin, isoparaffin, heavy liquid isoparaffin, paraffin, ozokerite, squalane, plant squalane, pristane, ceresin, squalene, petrolatum, microcrystalline wax, paraffin wax, montan wax, olefin oligomer, polyisobutylene, polybutene, hydrogenated polyisobutene, and the like.

Oils and fats liquid at room temperature: Examples include avocado oil, camellia oil, turtle oil, macadamia nut oil, corn oil, sunflower oil, mink oil, olive oil, rapeseed oil, egg yolk oil, sesame oil, persic oil, wheat germ oil, sasanqua oil, castor oil, linseed oil, safflower oil, grapeseed oil, cottonseed oil, perilla oil, soybean oil, peanut oil, tea seed oil, kaya oil, rice bran oil, Paulownia fortunei oil, Japanese tung oil, jojoba oil, germ oil, evening primrose oil, and the like.

Oils and fats solid at room temperature: Examples include cacao butter, coconut oil, beef tallow, mutton tallow, horse tallow, palm kernel oil, lard, bovine bone marrow fat, Rhus succedanea fruit wax, beef hoof fat, Rhus succedanea wax, hydrogenated coconut oil, hydrogenated palm oil, hydrogenated beef tallow, hydrogenated oil, hydrogenated castor oil, and the like.

Waxes: Examples include beeswax, candelilla wax, cotton wax, carnauba wax, bayberry wax, privet wax, spermaceti, montan wax, rice bran wax, kapok wax, sugarcane wax, lanolin, lanolin acetate, liquid lanolin, isopropyl lanolate, reduced lanolin, hydrogenated lanolin, hexyl lanolate, jojoba wax, shellac wax, POE lanolin alcohol ether, POE lanolin alcohol acetate, POE cholesterol ether, polyethylene glycol lanolin fatty acid, POE hydrogenated lanolin alcohol ether, and the like.

Lower alcohols: Examples include ethanol, isopropanol, and the like.

Higher alcohols: Examples include straight-chain alcohols such as lauryl alcohol, cetyl alcohol, stearyl alcohol, behenyl alcohol, myristyl alcohol, oleyl alcohol, cetostearyl alcohol, and the like; and branched alcohols such as monostearyl glycerin ether (batyl alcohol), 2-decyltetradecinol, lanolin alcohol, cholesterol, phytosterol, hexyldodecanol, isostearyl alcohol, octyldecanol, and the like.

Polyhydric alcohols: Examples include propylene glycol, ethylene glycol, dipropylene glycol, diethylene glycol, 1,3-butylene glycol, glycerin, and the like.

Higher fatty acids: Examples include lauric acid, myristic acid, palmitic acid, stearic acid, isostearic acid, and the like.

Esters: Examples include isopropyl myristate, cetyl octanate, octyl dodecyl myristate, isopropyl palmitate, isooctyl palmitate, butyl stearate, hexyl laurate, myristyl myristate, decyl oleate, hexyldecyl dimethyloctanate, cetyl lactate, myristyl lactate, octyl dodecyl lactate, lanolin acetate, isocetyl stearate, isocetyl isostearate, cholesteryl 1,2-hydroxystearate, phytosteryl 1,2-hydroxysterate, phytosteryl oleate, ethylene glycol di-2-ethylhexy, propylene glycol dicaprylate, dipentaerythritol fatty acid ester, N-alkyl glycol monoisostearate, neopentyl glycol dicapryate, diisostearyl malate, glycerin di-2-heptyl undecenoate, trimethyl propane tri-2-ethylhexanoate, trimethylolpropane triisostearate, pentaerythritol tetra-2-ethylhexanoate, glyceryl tri-2-ethylhexanoate, caprylic/capric/myristic/stearic triglyceride, trimethylolpropane triisostearate, cetyl 2-ethylhexanoate, 2-ethylhexyl palmitate, glyceryl trimyristate, glyceride tri-2-pentyl undecenoate, polyglyceryl diisostearate, polyglyceryl triisostearate, polyglyceryl tetraisostearate, diglyceryl triisostearate, diglyceryl tetraisostearate, erythrityl tri-2-ethylhexanoate, di-trimethylol propane tri-2-ethylhexanoate, isostearic/sebacic di-trimethylol propane oligo ester, castor oil fatty acid methyl ester, oleyl oleate, acetoglyceride, 2-heptylundecyl palmitate, diisobutyl adipate, adipic/2-ethylhexanoic/stearic oligoglyceride, 2-hexyldecanoic/sebacic di-oligoglyceride, N-laural-L-glutamic-2-octyl dodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, ethyl acetate, butyl acetate, triethyl citrate, and the like.

Silicone oil: Examples include straight chain silicone oils such as dimethyl polysiloxane, methylphenyl polysiloxane, methylhydrogen polysiloxane, and the like; branched silicone oils such as methyl trimethicone and the like; modified silicone oils such as polyether-modified silicone oil, polyglycerin-modified silicone oil, amino-modified silicone oil, polyether polyalkyl-modified silicone oil, and the like; and cyclic silicone oils such as octamethyl cyclotetrasiloxane, decamethyl cyclopentasiloxane, dodecamethyl cyclohexasiloxane, tetrahydrotetramethyl cyclotetrasiloxane, and the like.

Fluorinated solvents: Examples include perfluoro decalin, methyl perfluoro butyl ether, methyl perfluoro isobutyl ether, ethyl perfluoro butyl ether, ethyl perfluoro isobutyl ether, and the like.

Surfactants: Examples include anionic surfactants such as sodium N-alkyloyl methyl taurine, potassium stearate, sodium lauryl sulfate, sodium laureth-12 sulfate and the like; cationic surfactants such as quaternium-18 and the like; amphoteric surfactants such as coco-betaine, lauryl betaine, and the like; and nonionic surfactants such as PEG-2 hydrogenated castor oil, glyceryl stearate, and the like.

Humectants: Examples include polyethylene glycol, sorbitol, sodium lactate, sodium 2-pyrrolidone-5-carboxylic acid, sodium hyaluronate, betaine, and the like.

Thickeners: Examples include quince seed gum, xanthan gum, carboxymethyl cellulose sodium, carboxyvinyl polymer, bentonite, and the like.

Film-forming agents: Examples include polyvinyl alcohol, polyvinyl pyrrolidone, nitrocellulose, trimethyl siloxy silicate, acryl silicone, and the like.

Medicinal agents: Examples include skin lighteners such as vitamin C types, arbutin, kojic acid, ellagic acid, lucinol and the like; anti-wrinkle agents such as vitamin A types, α-hydroxy acids and the like; anti-inflammatory agents such as β-glycyrrhizic acid, dipotassium glycyrrhizinate, azulene, and the like; anti-comedogenic agents such as sulfur, salicylic acid, resorcinol, and the like; and herbal drugs such as witch hazel extract, aloe extract, chamomile extract, essence of eucalyptus, essence of calamus, essence of clove, tea tree oil, essence of rosemary, essence of angelica, hydrangea extract, and the like.

UV light absorbers: Examples include ethyl p-methoxycinnamate, isopropyl p-methoxycinnamate, octyl p-methoxycinnamate, 2-ethoxyethyl p-methoxycinnamate, sodium p-methoxycinnamate, p-aminobenzoic acid, ethyl p-aminobenzoic acid, 2-hydroxy-4-methoxy benzophenone, dihydroxymethoxy benzophenone, 2,4-dihydroxy benzophenone, octyl salicylate, myristyl salicylate, methyl salicylate, 4-t-butyl-4′-methoxy dibenzoyl methane, and the like.

Antiseptics: Examples include benzoic acid, benzoates, salicylic acid, salicylates, phenol, sorbic acid, sorbates, dehydroacetic acid, dehydroacetates, parabens, chloro-cresol, hexachlorophene, resorcinol, isopropyl methyl phenol, orthophenyl phenol, chlorhexidine chloride, chlorhexidine gluconate, alkyl isoquinolinium bromide, trichlorocarbanilide, halocarban, photosensitizing dye No. 201, phenoxyethanol, triclosan, cetylpyridinium chloride, benzalkonium chloride, benzethonium chloride, and the like.

Metal ion chelating agents: Examples include edetic acid and the like.

Fragrances: Examples include limonene, β-caryophyllene, cis-3-hexenol, linalool, farnesol. β-phenylethyl alcohol, 2,6-nonadienal, citral, α-hexyl cinnamic aldehyde, β-ionone, l-carvone, cycloheptadecanone, linalyl acetate, benzyl benzoate, γ-undecalactone, eugenol, rose oxides, indoles, phenylacetaldehyde dimethyl acetal, auranthiol, and the like.

The present invention has been described in detail above based on embodiments. However, the present invention is by no means limited to the above embodiments. The invention is capable of other embodiments and of being practiced or of being carried out in various ways.

### EXAMPLES

The present invention is described in greater detail below through preferred examples, but the present invention is by no means limited thereto.

### Test Method for Dispersibility

Samples were prepared by placing 2.4 g of decamethyl cyclopentasiloxane and 0.1 g of the silicone treated powder (or untreated powder) obtained in the following examples and comparative examples into a 3 mL polystyrene test tube. Sonication was performed on the obtained samples for 20 minutes, the samples were let stand for 12 hours, and the height of precipitate in the samples was measured.

### Test Method for Water Repellency

Samples were prepared by placing suitable amounts of water and decamethyl cyclopentasiloxane, and 0.02 g of the silicone treated powder (or untreated powder) obtained in the following examples and comparative examples into a 30 mL glass sample tube. The samples were shaken well by hand and let stand for 1 hour. Then the undisturbed samples were given a numerical score using the criteria shown in Table 1.

TABLE 1

| Score | Condition after standing undisturbed |
|---|---|
| 5 | The water and decamethyl cyclopentasiloxane separated into |
| | 2 phases, and all powder was present in the decamethyl |
| | cyclopentasiloxane phase. |
| 4 | The water and decamethyl cyclopentasiloxane separated into 2 |
| | phases, and a very small amount of powder was present in the |
| | aqueous phase. |
| 3 | The water and decamethyl cyclopentasiloxane separated into 2 |
| | phases, and about half the powder was present in the |
| | aqueous phase. |
| 2 | The decamethyl cyclopentasiloxane phase was finely divided and |
| | present in the aqueous phase, and about half the powder was |
| | present in the aqueous phase. |
| 1 | All the powder was present in the aqueous phase. |

### Test Methods for Coverage and Texture

The silicone treated powders (or untreated powders) obtained in the following examples and comparative examples were evaluated by 7 test subjects for coverage and texture when the powder was applied to the forearm using the criteria shown in Table 2. Each of the treated powders from the examples and comparative examples was evaluated using the untreated powder as a reference sample.

TABLE 2

| Score | Coverage | Texture |
|---|---|---|
| 5 | Much better than reference sample. | Much better than reference |
| | | sample. |
| 4 | Better than reference sample. | Better than reference sample. |
| 3 | Same as reference sample. | Same as reference sample. |
| 2 | Worse than reference sample. | Worse than reference sample. |
| 1 | Much worse than reference sample. | Much worse than reference |
| | | sample. |

### Synthesis Examples 1-6

Using the compositions shown in Table 3, mercapto-modified dimethyl polysiloxane (KF-2001™ manufactured by Shin-Etsu Chemical Co., Ltd., mercapto-propylmethylsiloxane units: 4 mol %, weight-average molecular weight: 20,000), monomer component, solvent, and polymerization initiator were placed in a 225 mL glass bottle to make a total of 160.5 g (synthesis examples 2 and 4) or 162.4 g (synthesis examples 1, 3, 5, and 6), and nitrogen gas was bubbled through the mixture for 10 minutes. Then the container was sealed, and polymerization was performed with stirring in a constant temperature bath at 65° C. After 24 hours, the container was removed from the constant temperature bath and cooled to room temperature. The polymer solution obtained thereby was dripped into water with stirring and the precipitate was recovered. The precipitate was dried at room temperature to obtain the (meth)acrylic-grafted silicone polymer.

TABLE 3
(Units: Parts by weight)

| | | Synthesis | Synthesis | Synthesis | Synthesis | Synthesis | Synthesis |
|---|---|---|---|---|---|---|---|
| Component | | Example 1 | Example 2 | Example 3 | Example 4 | Example 5 | Example 6 |
| Silicone | Mercapto-modified dimethyl polysiloxane | 60.00 | 67.50 | 40.00 | 60.00 | 60.00 | 45.00 |
| polymer | | | | | | | |
| Monomer | Methacrylate | 11.44 | 9.30 | 17.20 | | | |
| component | Methyl methacrylate | 28.56 | 23.20 | | | | |
| | Ethyl methacrylate | | | 42.80 | | | |
| | Isobutyl methacrylate | | | | 40.00 | 35.00 | |
| | Methacrylic acid | | | | | 5.00 | |
| | Cyclohexyl methacrylate | | | | | | 55.00 |
| Initiator | 2,2′-azo bis (2-methylbutyronitrile)* | | 0.60 | | 0.60 | | |
| | Dimethyl-2,2′-azo bis (2-methyl propionate)** | 0.60 | | 0.60 | | 0.60 | 0.60 |
| Solvent | Methyl ethyl ketone | 102.40 | 100.00 | 102.40 | 100.00 | 102.40 | 102.40 |

*Wako Pure Chemical Industries, Ltd., V-59 ™
**Wako Pure Chemical Industries, Ltd. Brand name: V-60 ™

### Examples 1-14, Comparative Examples 1-8

The various components were added to a 225 mL glass bottle based on the compositions shown in Table 4 to make a total of 105 g, stirred with a high speed disperser, and dissolved. Next, 45 g of titanium dioxide powder (MT500B™ manufactured by Tayca Corporation) was slowly added while stirring with a high speed disperser. Then, stirring was performed with the high speed disperser for 1 hour at the speed shown in Table 4. The obtained liquid dispersion was placed in an aluminum cup, and heated and dried on a hot plate from 50° C. to 100° C. to obtain silicone-treated titanium dioxide powder. Untreated titanium oxide powder (MT500B™ manufactured by Tayca Corporation) was used as comparative example 1 (reference sample).

Using the aforementioned assessment methods, the powders of examples 1-14 and comparative examples 1-8 were evaluated for dispersibility, water repellency, coverage and texture. The results are shown in Table 4.

TABLE 4

| | | | | Polymer component | | | | | | | |
|---|---|---|---|---|---|---|---|---|---|---|---|
| | | Solvent | | | | Amt. (parts | Speed | Dispersibility | Water | | |
| | | Type | Wt. (g) | Type | Wt. (g) | by weight)* | (rpm) | (mm) | repellency | Coverage | Texture |
| Examples | 1 | A | 103.65 | S.E. 1^{†} | 1.35 | 3 | 2000 | 9.0 | 5 | 4.4 | 4.6 |
| | 2 | A1 | 103.65 | S.E. 1 | 1.35 | 3 | 2000 | 9.0 | 5 | 3.5 | 3.7 |
| | 3 | A2 | 103.65 | S.E. 1 | 1.35 | 3 | 2000 | 10.0 | 5 | 3.3 | 3.5 |
| | 4 | B | 103.65 | S.E. 1 | 1.35 | 3 | 2000 | 8.5 | 5 | 4.0 | 4.0 |
| | 5 | A | 104.95 | S.E. 1 | 0.05 | 0.1 | 2000 | 17.0 | 3 | 3.4 | 3.5 |
| | 6 | A | 104.77 | S.E. 1 | 0.23 | 0.5 | 2000 | 14.0 | 3 | 3.8 | 3.9 |
| | 7 | A | 104.55 | S.E. 1 | 0.45 | 1 | 2000 | 14.0 | 4 | 3.7 | 4.1 |
| | 8 | A | 102.75 | S.E. 1 | 2.25 | 5 | 2000 | 6.0 | 5 | 3.8 | 3.9 |
| | 9 | A | 100.50 | S.E. 1 | 4.50 | 10 | 1500 | 5.0 | 5 | 4.0 | 3.9 |
| | 10 | A | 103.65 | S.E. 2 | 1.35 | 3 | 2000 | 7.0 | 5 | 4.1 | 4.0 |
| | 11 | A | 103.65 | S.E. 3 | 1.35 | 3 | 2000 | 7.0 | 5 | 4.1 | 4.0 |
| | 12 | A | 103.65 | S.E. 4 | 1.35 | 3 | 2000 | 9.0 | 5 | 3.9 | 4.2 |
| | 13 | A | 103.65 | S.E. 5 | 1.35 | 3 | 2000 | 16.0 | 5 | 3.6 | 3.9 |
| | 14 | A | 103.65 | S.E. 6 | 1.35 | 3 | 2000 | 9.0 | 5 | 3.9 | 4.2 |
| Comparative | 1 | — | — | — | — | — | — | 24.0 | 2 | 3.0 | 3.0 |
| Examples | 2 | C | 103.65 | S.E. 1 | 1.35 | 3 | 1500 | 14.5 | 3 | 2.4 | 2.4 |
| | 3 | D | 103.65 | S.E. 1 | 1.35 | 3 | 1500 | 16.0 | 2 | 2.9 | 2.6 |
| | 4 | E | 103.65 | S.E. 1 | 1.35 | 3 | 2000 | 18.0 | 2 | 3.0 | 3.0 |
| | 5 | A | 103.50 | F | 1.50** | 1 | 2000 | 11.5 | 5 | 3.5 | 3.7 |
| | 6 | A | 100.50 | F | 4.50*** | 3 | 2000 | 10.0 | 5 | 3.5 | 3.7 |
| | 7 | A | 104.65 | G | 0.45 | 1 | 2000 | 10.0 | 2 | 3.5 | 3.6 |
| | 8 | A | 103.65 | G | 1.35 | 3 | 2000 | 7.5 | 5 | 3.4 | 3.4 |

^{†}S.E. = synthesis example,
*Amount of polymer (parts by weight) per 100 parts by weight of titanium dioxide powder,
**Polymer solids = 0.45 g,
***Polymer solids = 1.35 g,
A: decamethyl cyclopentasiloxane (KF-995 ™ manufactured by Shin-Etsu Chemical Co., Ltd.), A1: 3:1(weight ratio) mixed solution of decamethyl cyclopentasiloxane (KF-995 ™ manufactured by Shin-Etsu Chemical Co., Ltd.) and methyl ethyl ketone, A2: 1:1(weight ratio) mixed solution of decamethyl cyclopentasiloxane (KF-995 ™ manufactured by Shin-Etsu Chemical Co., Ltd.) and methyl ethyl ketone, B: dimethyl polysiloxane (KF-96A-1cs ™ manufactured by Shin-Etsu Chemical Co., Ltd.), C: butyl acetate, D: isopropanol, E: methyl ethyl ketone, F: silicone-grafted (meth)acrylic polymer (polymer having silicone moiety on (meth)acrylic polymer main chain) (KP-545 ™ manufactured by Shin-Etsu Chemical Co., Ltd., 30% (wt %) decamethyl cyclopentasiloxane solution), G: silicone-grafted (meth)acrylic polymer (KP-561P ™ manufactured by Shin-Etsu Chemical Co., Ltd.)

### Examples 15-16 and Comparative Examples 9-13

The various components were added to a 225 mL glass bottle based on the compositions shown in Table 5 to make a total of 105 g, stirred with a high speed disperser, and dissolved. Next, 45 g of sericite powder (FSE™ manufactured by Sanshin Mining Ind. Co., Ltd.) was slowly added while stirring with a high speed disperser. Then, the same procedure as in example 1 was performed except that stirring with the high speed disperser was carried out at 1500 rpm. The obtained liquid dispersion was placed in an aluminum cup, and heated and dried on a hot plate from 50° C. to 100° C. to obtain silicone-treated sericite powder. Untreated sericite powder (FSE™ manufactured by Sanshin Mining Ind. Co., Ltd.) was used as comparative example 9 (reference sample).

### Comparative Example 14

Sericite powder (SI-FSE™ manufactured by Miyoshi Kasei, Inc.) treated with methyl hydrogen polysiloxane was used.

### Comparative Example 15

Sericite powder (SA-FSE™ manufactured by Miyoshi Kasei, Inc.) treated with dimethyl polysiloxane was used.

Using the aforementioned assessment methods, the powders of examples 15-16 and comparative examples 9-15 were evaluated for dispersibility, water repellency, coverage and texture. The results are shown in table 5.

TABLE 5

| | | | | Polymer component | | | | | | | |
|---|---|---|---|---|---|---|---|---|---|---|---|
| | | Solvent | | | | Amt. (parts | Speed | Dispersibility | Water | | |
| | | Type | Wt. (g) | Type | Wt. (g) | by weight)* | (rpm) | (mm) | repellency | Coverage | Texture |
| Examples | 15 | A | 104.55 | S.E. 1^{†} | 0.45 | 1 | 1500 | 8.0 | 5 | 3.4 | 3.7 |
| | 16 | A | 103.65 | S.E. 1 | 1.35 | 3 | 1500 | 5.5 | 5 | 4.0 | 4.3 |
| Comparative | 9 | — | — | — | — | — | — | 17.5 | 2 | 3.0 | 3.0 |
| Examples | 10 | A | 103.50 | F | 1.50** | 1 | 1500 | 6.5 | 5 | 3.2 | 3.4 |
| | 11 | A | 100.50 | F | 4.50*** | 3 | 1500 | 7.5 | 5 | 3.5 | 3.5 |
| | 12 | A | 104.55 | G | 0.45 | 1 | 1500 | 9.0 | 5 | 3.2 | 3.4 |
| | 13 | A | 103.65 | G | 1.35 | 3 | 1500 | 6.0 | 5 | 3.6 | 3.8 |
| | 14 | — | — | H | — | — | — | 7.0 | 5 | 2.7 | 2.8 |
| | 15 | — | — | I | — | — | — | 6.0 | 5 | 3.2 | 3.1 |

^{†}S.E. = synthesis example,
*Amount of polymer (parts by weight) per 100 parts by weight of sericite powder,
**Polymer solids = 0.45 g,
***Polymer solids = 1.35 g,
A, F, & G: Same as in Table 4, H: methyl hydrogen polysiloxane, I: dimethyl polysiloxane

### Example 17, Comparative Examples 16-18

The various components were added to a 225 mL glass bottle based on the compositions shown in Table 6 to make a total of 105 g, stirred with a high speed disperser, and dissolved. Next, 45 g of cosmetic powder was slowly added while stirring with a high speed disperser. Then, the same procedure as in example 1 was performed except that stirring with the high speed disperser was carried out at 1000 rpm. The obtained liquid dispersion was placed in an aluminum cup, and heated and dried on a hot plate from 50° C. to 100° C. to obtain silicone-treated powder. The term “cosmetic powder” is a mixture of 65.1 parts by weight of sericite (TKC™ manufactured by Toshiki Pigment Co., Ltd.), 32.6 parts by weight of talc (JA-46R™ manufactured by Asada Milling Co., Ltd.), 1.1 parts by weight yellow iron oxide (LL-XLO™ manufactured by Titan Kogyo, Co., Ltd.), 0.6 parts by weight red iron oxide (R-516L™ manufactured by Titak Kogyo Co., Ltd.), and 0.6 parts by weight black iron oxide (BL-100™ manufactured by Titan Kogyo Co., Ltd.) This untreated cosmetic powder was used as comparative example 16 (reference sample).

Using the aforementioned assessment methods, the powders of example 17 and comparative examples 16-18 were evaluated for dispersibility, water repellency, coverage and texture. The results are shown in table 6.

TABLE 6

| | | | | Polymer component | | | | | | | |
|---|---|---|---|---|---|---|---|---|---|---|---|
| | | Solvent | | | | Amt. (parts by | Speed | Dispersibility | Water | | |
| | | Type | Wt. (g) | Type | Wt. (g) | weight)* | (rpm) | (mm) | repellency | Coverage | Texture |
| Examples | 17 | A | 103.65 | S.E. 1^{†} | 1.35 | 3 | 1000 | 7.0 | 5 | 3.9 | 3.9 |
| Comparative | 16 | — | — | — | — | — | — | 14.0 | 1 | 3.0 | 3.0 |
| Examples | 17 | A | 100.50 | F | 4.50** | 3 | 1000 | 7.0 | 5 | 2.9 | 2.8 |
| | 18 | A | 103.65 | G | 1.35 | 3 | 1000 | 7.0 | 5 | 3.1 | 3.6 |

^{†}S.E. = synthesis example,
*Amount of polymer (parts by weight) per 100 parts by weight of cosmetic powder,
**Polymer solids = 1.35 g,
A, F, & G: Same as in Table 4

### Manufacturing Examples A-F

Referring to the compositions in Table 7, solvent and polymer component were placed in a glass bottle and dissolved. Next, powder was slowly added while stirring with a high speed disperser and stirring was continued. The obtained liquid dispersion was placed in an aluminum cup, and heated and dried on a hot plate from 50° C. to 100° C. to obtain silicone-treated powder.

TABLE 7
(Units: Parts by weight)

| | | Mfg. Ex. A | Mfg. Ex. B | Mfg. Ex. C | Mfg. Ex. D | Mfg. Ex. E | Mfg. Ex. F |
|---|---|---|---|---|---|---|---|
| Component | | (Example) | (Example) | (Comp. Ex.) | (Comp. Ex.) | (Comp. Ex.) | (Comp. Ex.) |
| Solvent | Decamethyl | 69.1 | 51.6 | 67.0 | 48.3 | 48.5 | 51.6 |
| | cyclopenta-siloxane⁽¹⁾ | | | | | | |
| Polymer | Synthesis example 1 | 0.9 | 1.4 | | | | |
| component | Silicone-grafted (meth) acrylic | | | 3.0 | 4.7 | | |
| | polymer⁽²⁾ | | | | | | |
| | Silicone-grafted (meth) acrylic | | | | | 1.5 | 1.4 |
| | polymer⁽³⁾ | | | | | | |
| Powder | Sericite powder | 30.0 | | 30.0 | | 50.0 | |
| | Titanium dioxide powder | | 36.0 | | 36.0 | | 36.0 |
| | Yellow iron oxide | | 8.0 | | 8.0 | | 8.0 |
| | Red iron oxide | | 2.0 | | 2.0 | | 2.0 |
| | Black iron oxide | | 1.0 | | 1.0 | | 1.0 |

⁽¹⁾KF-995 ™ manufactured by Shin-Etsu Chemical Co., Ltd.
⁽²⁾KP-545 ™ manufactured by Shin-Etsu Chemical Co., Ltd. (polymer having silicone moiety grafted onto a (meth)acrylic polymer main chain, 30% decamethyl cyclopentasiloxane solution)
⁽³⁾KP-561P ™ manufactured by Shin-Etsu Chemical Co., Ltd. (polymer having silicone moiety grafted onto a (meth)acrylic polymer main chain)

### Example 18

As shown in Table 8, a powdery foundation was prepared using the silicone-treated powders of manufacturing examples A and B. First, components (1) to (8) were mixed together with a mixer, and then components (9) and (10) were added and mixed thoroughly. The mixture that was removed from the mixer was packed into a conventional metal container and pressed with a load of 1,000 kgf to produce a powdery foundation.

TABLE 8

| | | Parts by |
|---|---|---|
| | Component | weight |
| (1) | Zinc myristate | 2.0 |
| (2) | Silicone-treated powder from Manufacturing Example A | 47.1 |
| (3) | Nylon | 8.0 |
| (4) | Boron nitride | 3.0 |
| (5) | Synthetic brown mica | 5.0 |
| (6) | Silicate anhydride | 2.0 |
| (7) | Methyl hydrogen diene polysiloxane-treated talc powder | 10.5 |
| (8) | Methylparaben | 0.2 |
| (9) | Silicone-treated powder from Manufacturing Example B | 12.1 |
| (10) | Trioctanoin | 10.0 |

### Comparative Example 19

A powdery foundation was prepared as shown in Table 9. First, components (1) to (8) were mixed together with a mixer, and then components (9) to (13) were added and mixed thoroughly. The mixture that was removed from the mixer was packed into a conventional metal container and pressed with a load of 1,000 kgf to produce a powdery foundation.

TABLE 9

| | | Parts by |
|---|---|---|
| | Component | weight |
| (1) | Zinc myristate | 2.0 |
| (2) | Sericite powder | 46.5 |
| (3) | Nylon | 8.0 |
| (4) | Boron nitride | 3.0 |
| (5) | Synthetic brown mica | 5.0 |
| (6) | Silicate anhydride | 2.0 |
| (7) | Methyl hydrogen diene polysiloxane-treated talc powder | 10.5 |
| (8) | Methylparaben | 0.2 |
| (9) | Titanium dioxide | 9.0 |
| (10) | Yellow iron oxide | 2.1 |
| (11) | Red iron oxide | 0.5 |
| (12) | Black iron oxide | 0.3 |
| (13) | Trioctanoin | 11.0 |

### Comparative Example 20

As shown in Table 10, a powdery foundation was prepared using the silicone-treated powders of manufacturing examples C and D. First, components (1) to (8) were mixed together with a mixer, and then components (9) and (10) were added and mixed thoroughly. The mixture that was removed from the mixer was packed into a conventional metal container and pressed with a load of 1,000 kgf to produce a powdery foundation.

TABLE 10

| | | Parts by |
|---|---|---|
| | Component | weight |
| (1) | Zinc myristate | 2.0 |
| (2) | Silicone-treated powder from Manufacturing Example C | 47.1 |
| (3) | Nylon | 8.0 |
| (4) | Boron nitride | 3.0 |
| (5) | Synthetic brown mica | 5.0 |
| (6) | Silicate anhydride | 2.0 |
| (7) | Methyl hydrogen diene polysiloxane-treated talc powder | 10.5 |
| (8) | Methylparaben | 0.2 |
| (9) | Silicone-treated powder from Manufacturing Example D | 12.1 |
| (10) | Trioctanoin | 10.0 |

### Comparative Example 21

As shown in Table 11, a powdery foundation was prepared using the silicone-treated powders of manufacturing examples E and F. First, components (1) to (8) were mixed together with a mixer, and then components (9) and (10) were added and mixed thoroughly. The mixture that was removed from the mixer was packed into a conventional metal container and pressed with a load of 1,000 kgf to produce a powdery foundation.

TABLE 11

| | | Parts by |
|---|---|---|
| | Component | weight |
| (1) | Zinc myristate | 2.0 |
| (2) | Silicone-treated powder from Manufacturing Example E | 47.1 |
| (3) | Nylon | 8.0 |
| (4) | Boron nitride | 3.0 |
| (5) | Synthetic brown mica | 5.0 |
| (6) | Silicate anhydride | 2.0 |
| (7) | Methyl hydrogen diene polysiloxane-treated talc powder | 10.5 |
| (8) | Methylparaben | 0.2 |
| (9) | Silicone-treated powder from Manufacturing Example F | 12.1 |
| (10) | Trioctanoin | 10.0 |

The powdery foundations obtained in the examples and comparative examples were evaluated by 5 test subjects for coverage and texture when the powdery foundation was applied to the forearm using the criteria shown in Table 12. The powdery foundation from comparative example 19 was used as a reference sample.

TABLE 12

| Score | Coverage | Texture |
|---|---|---|
| 5 | Much better than reference sample. | Much better than reference |
| | | sample. |
| 4 | Better than reference sample. | Better than reference sample. |
| 3 | Same as reference sample. | Same as reference sample. |
| 2 | Worse than reference sample. | Worse than reference sample. |
| 1 | Much worse than reference sample. | Much worse than reference |
| | | sample. |

Table 13 shows the results of the evaluation of the powdery foundations obtained in example 18 and comparative examples 19 to 21.

TABLE 13

| | Coverage | Texture |
|---|---|---|
| Example 18 | 3.8 | 3.6 |
| Comparative Example 19 | 3.0 | 3.0 |
| Comparative Example 20 | 3.1 | 3.3 |
| Comparative Example 21 | 3.3 | 3.1 |

### Example 19

As shown in Table 14, a powdery foundation was prepared using the silicone-treated powder of manufacturing example A. First, components (1) to (7) were mixed together with a mixer, and then components (8) to (11) were added and mixed thoroughly. The mixture that was removed from the mixer was packed into a conventional metal container and pressed with a load of 1,000 kgf to produce a powdery foundation.

TABLE 14

| | | Parts by |
|---|---|---|
| | Component | weight |
| (1) | Zinc myristate | 2.0 |
| (2) | Silicone-treated sericite powder from Manufacturing | 50.0 |
| | Example A | |
| (3) | Nylon | 8.0 |
| (4) | Synthetic brown mica | 8.0 |
| (5) | Silicate anhydride | 2.0 |
| (6) | Methyl hydrogen diene polysiloxane-treated talc powder | 11.5 |
| (7) | Methylparaben | 0.2 |
| (8) | Methyl hydrogen diene polysiloxane-treated | 7.0 |
| | titanium dioxide powder | |
| (9) | Yellow iron oxide, red iron oxide, black iron oxide | 2.3 |
| (10) | Squalene | 4.5 |
| (11) | Trioctanoin | 4.5 |

### Comparative Example 22

A powdery foundation was prepared as shown in Table 15. First, components (1) to (7) were mixed together with a mixer, and then components (8) to (11) were added and mixed thoroughly. The mixture that was removed from the mixer was packed into a conventional metal container and pressed with a load of 1,000 kgf to produce a powdery foundation.

TABLE 15

| | | Parts by |
|---|---|---|
| | Component | weight |
| (1) | Zinc myristate | 2.0 |
| (2) | Sericite powder | 50.0 |
| (3) | Nylon | 8.0 |
| (4) | Synthetic brown mica | 8.0 |
| (5) | Silicate anhydride | 2.0 |
| (6) | Methyl hydrogen diene polysiloxane-treated talc powder | 10.5 |
| (7) | Methylparaben | 0.2 |
| (8) | Methyl hydrogen diene polysiloxane-treated | 7.0 |
| | titanium dioxide powder | |
| (9) | Yellow iron oxide, red iron oxide, black iron oxide | 2.3 |
| (10) | Squalene | 5.0 |
| (11) | Trioctanoin | 5.0 |

The powdery foundations obtained in manufacturing example 19 and comparative example 22 were applied to the face, and the coverage, blending properties, and loss of fresh appearance were evaluated by 10 test subjects. The coverage, blending properties, and loss of fresh appearance over time were scored on a 5-step scale using the criteria shown in Table 16. Table 17 shows the results of the evaluation of the powdery foundations obtained in example 19 and comparative example 22.

TABLE 16

| | | | Loss of fresh appearance |
|---|---|---|---|
| Score | Coverage | Blending | over time |
| 5 | Excellent | Excellent | Very unlikely to occur |
| 4 | Good | Good | Unlikely to occur |
| 3 | Average | Average | Average |
| 2 | Poor | Poor | Likely to occur |
| 1 | Very poor | Very poor | Very likely to occur |

TABLE 17

| | | | Loss of fresh |
|---|---|---|---|
| | Coverage | Blending | appearance over time |
| Example 19 | 3.9 | 3.9 | 3.5 |
| Comparative example 22 | 3.2 | 3.1 | 3.1 |

### Manufacturing Examples G-J

Parts by weight of decamethyl cyclopentasiloxane and 3 parts by weight of the graft polymer from synthesis example 1 were placed in a glass bottle, stirred with a high speed disperser, and dissolved. Next, 100 parts by weight of powder shown in Table 18 were slowly added while stirring with a high speed disperser, and stirring was continued. The obtained liquid dispersion was placed in an aluminum cup, and heated and dried on a hot plate from 50° C. to 100° C. to obtain silicone-treated powder.

TABLE 18

| | | Mfg. Ex. G | Mfg. Ex. H | Mfg. Ex. I | Mfg. Ex. J |
|---|---|---|---|---|---|
| Component | | (Example) | (Example) | (Example) | (Example) |
| Decamethyl cyclopentasiloxane⁽¹⁾ | | 97.0 | 97.0 | 97.0 | 97.0 |
| Silicone polymer from synthesis example 1 | | 3.0 | 3.0 | 3.0 | 3.0 |
| Powder | Titanium dioxide powder | 100.0 | | | |
| | Red iron oxide | | 100.0 | | |
| | Yellow iron oxide | | | 100.0 | |
| | Black iron oxide | | | | 100.0 |

⁽¹⁾Same as Table 7

### Example 20

A liquid foundation was prepared using the silicone-treated powders of manufacturing examples G-J as shown in Table 19. First, components (11) and (12) were mixed at 60° C., and after dissolution the mixture was cooled to room temperature, components (1) to (10) were dripped in and mixed to prepare the oil-phase. Then, components (13) to (16) were dripped in and mixed to prepare the aqueous phase. Next, the aqueous phase was dripped into the oil phase at room temperature and mixed to obtain an emulsion. The emulsion was placed in a conventional plastic container to make a water-in-oil type liquid foundation.

TABLE 19

| | | Parts by |
|---|---|---|
| | Component | weight |
| (1) | Natural vitamin E | 0.05 |
| (2) | Isotridecyl isononanoate | 7 |
| (3) | Polyoxyethylene-methyl polysiloxane copolymer | 4 |
| (4) | Methyl polysiloxane | 2 |
| (5) | Dimethyl distearyl ammonium hectorite | 0.3 |
| (6) | Silicone-treated titanium dioxide powder | 9.24 |
| | from Mfg. Ex. G | |
| (7) | Silicone-treated red iron oxide from Mfg. Ex. H | 0.54 |
| (8) | Silicone-treated yellow iron oxide from Mfg. Ex. I | 2.13 |
| (9) | Silicone-treated black iron oxide from Mfg. Ex. J | 0.26 |
| (10) | Phenoxyethanol | 0.3 |
| (11) | Methyl polysiloxane | 4.94 |
| (12) | Decamethyl cyclopentasiloxane | 10.13 |
| (13) | Purified water | 55.01 |
| (14) | Carboxymethyl cellulose sodium | 0.6 |
| (15) | Magnesium sulfate | 0.5 |
| (16) | 1,2-pentanediol | 3 |

### Comparative Example 23

A liquid foundation was prepared as shown in Table 20. First, components (11) and (12) were mixed at 60° C., and after dissolution the mixture was cooled to room temperature, components (1) to (10) were dripped in and mixed to prepare the oil-phase. Then, components (13) to (16) were dripped in and mixed to prepare the aqueous phase. Next, the aqueous phase was dripped into the oil phase at room temperature and mixed to obtain an emulsion. The emulsion was placed in a conventional plastic container to make a water-in-oil type liquid foundation.

TABLE 20

| | | Parts by |
|---|---|---|
| | Component | weight |
| (1) | Natural vitamin E | 0.05 |
| (2) | Isotridecyl isononanoate | 7 |
| (3) | Polyoxyethylene-methyl polysiloxane copolymer | 4 |
| (4) | Methyl polysiloxane | 2 |
| (5) | Dimethyl distearyl ammonium hectorite | 0.3 |
| (6) | Aluminum dimyristate-treated titanium dioxide powder | 9.11 |
| (7) | Aluminum dimyristate-treated red iron oxide | 0.53 |
| (8) | Aluminum dimyristate-treated yellow iron oxide | 2.10 |
| (9) | Aluminum dimyristate-treated black iron oxide | 0.26 |
| (10) | Phenoxyethanol | 0.3 |
| (11) | Methyl polysiloxane | 4.94 |
| (12) | Decamethyl cyclopentasiloxane | 10.13 |
| (13) | Purified water | 55.01 |
| (14) | Carboxymethyl cellulose sodium | 0.6 |
| (15) | Magnesium sulfate | 0.5 |
| (16) | 1,2-pentanediol | 3 |

The liquid foundations obtained in example 20 and comparative example 23 were applied to the face, and the coverage, blending properties, and loss of fresh appearance were evaluated by 10 test subjects. The same criteria were used as shown in Table 16. The liquid foundation of example 20 had better blending properties and retained a fresh appearance much longer than that of comparative example 23.

1. A treated powder comprising a (meth)acrylic-grafted silicone polymer, the treated powder obtained from a mixture containing a volatile silicone oil, a (meth)acrylic-grafted silicone polymer in which a monomer component comprising a (meth)acrylic series monomer has been grafted onto the main chain of a polysiloxane, and a powder.

2. The treated powder according to claim 1, wherein the content of (meth)acrylic-grafted silicone polymer in the mixture ranges from 0.1 to 10 parts by weight per 100 parts by weight of the powder.

3. The treated powder according to claim 1, wherein the monomer component comprising the (meth)acrylic series monomer comprises at least one type of (meth)acrylic series monomer selected from the group consisting of (meth)acrylic acid and (meth)acrylic acid esters.

4. The treated powder according to claim 1, wherein the (meth)acrylic-grafted silicone polymer comprises a polymerization product between a mercapto-modified silicone polymer represented by General Formula (1) below and a monomer component comprising at least one type of (meth)acrylic series monomer selected from the group consisting of (meth)acrylic acid and (meth)acrylic acid esters 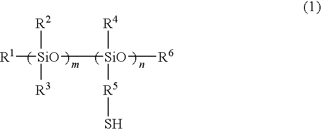
wherein
R¹, R², R³, R⁴, and R⁶ each independently represents a hydrogen atom, hydroxyl group, allyl group, alkyl group of 1 to 3 carbons, or a halogenated alkyl group of 1 to 3 carbons,
R⁵ represents an arylene group or alkylene group of 1 to 3 carbon atoms,
m is an integer from 10 to 540, and
n is an integer of 1 or more.

5. A cosmetic product containing the treated powder according to claim 1.
